(19) United States
(12) Patent Application Publication
(10) Pub. No.: US 2010/0000897 A1
(43) Pub. Date: Jan. 7, 2010

(54) FEMININE HYGIENE PRODUCTS HAVING A PSEUDOMOTIVE

(76) Inventors: Linda Marie Bumpass, Crestview Hills, KY (US); Margaret Henderson Hasse, Wyoming, OH (US); Peter Michael Searles, Mason, OH (US); Steven Ray Gilbert, Fairfield, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/166,935

(22) Filed: Jul. 2, 2008

(51) Int. Cl. A61B 19/00; B65D 85/00; G09F 3/00; A61F 13/15

(52) U.S. Cl. 206/440; 206/459.1; 206/459.5; 40/299.01; 604/385.02

(57) ABSTRACT

A feminine hygiene package. The feminine hygiene package may include a feminine hygiene product and a package enclosing the feminine hygiene product. The feminine hygiene product and/or package may include a pseudomotive thereon.

### TECHNICAL FIELD

The present application relates generally to feminine hygiene articles and packaging and more particularly relates to feminine hygiene articles and packaging having one or more pseudomotives.

### BACKGROUND OF THE INVENTION

Known feminine hygiene products, such as, e.g., sanitary napkin and tampon products, and other types of consumer products, generally have a limited amount of space on the product and/or packaging, and/or elsewhere to provide communication to the consumer via traditional two-dimensional graphics. Given such, demonstrating the benefits of or providing information relating to a better designed feminine hygiene article, such as, e.g., a sanitary napkin, a tampon, an applicator, or other elements, may present a challenge because of the space and size constraints. Therefore, there remains a need for an improved feminine hygiene article and/or packaging that can provide such benefits.

### SUMMARY OF THE INVENTION

A feminine hygiene package is provided. The feminine hygiene package can include a feminine hygiene product and a package, the package comprising a pseudomotive thereon.

A feminine hygiene product is further provided. The feminine hygiene produe can include a pseudomotive thereon.

Also provided is a pseudomotive, comprising a label or film. In certain embodiments, the label or film can include a first image of a feminine hygiene product; and a second image of the feminine hygiene product, wherein the first image and the second image are different. In certain embodiments, the pseudomotive can include a vision of a use, an application, and/or an insertion of the feminine hygiene product. In addition, or alternatively, the pseudomotive can include instructions for use of the feminine hygiene product.

### BRIEF DESCRIPTION OF THE DRAWINGS

FIG. 1 is a perspective view of a tampon within an applicator.

FIG. 2 is a perspective view of an alternative applicator.

FIG. 3 is a perspective view of a tampon wrapper.

FIG. 4 is a perspective view of a tampon package and a tampon package insert.

FIG. 5 is a perspective view of the layers of a lenticular label or film.

FIG. 6 is a side plan view of a lenticular lens.

FIGS. 7A-7C are plan views showing instructions for use.

FIG. 8 is a perspective view of the layers of a holographic label or film.

FIG. 9 is a perspective view of a lenticular applicator with a lenticular label applied thereto as is described herein.

FIG. 10 is a side cross-sectional view of a lenticular applicator with a lenticular film applied thereto as is described herein.

FIG. 11 is a perspective view of a lenticular kit with a lenticular applicator, a lenticular package, a lenticular wrapper, and a lenticular packaging insert.

FIG. 12 is a perspective view of a holographic applicator with a holographic label applied thereto as is described herein.

FIG. 13 is a side cross-sectional view of a holographic applicator with a holographic film applied thereto as is described herein.

FIG. 14 is a perspective view of a holographic kit with a holographic applicator, a holographic package, a holographic wrapper, and a holographic packaging insert.

FIG. 15 is a plan view of a digital tampon wrapper with a multidimensional communication thereon.

FIG. 16 is a perspective view of a feminine hygiene kit with tampons and sanitary napkins therein.

### DETAILED DESCRIPTION

The present application may encompass feminine hygiene products and packaging that can include one or more pseudomotives, such as, e.g., one or more lenticular images, holographic images, and/or combinations thereof. As a result, the feminine hygiene article, the applicator, the wrapper, the package, and/or other elements and products may provide improved and coordinated communication to the consumer as compared to traditional two-dimensional graphics and imaging. In certain embodiments, the feminine hygiene article, applicator, and/or associated packaging may provide multiple messages, images, and/or other forms of communication to the consumer. In addition, or alternatively, the feminine hygiene article, applicator, and/or the packaging may present information to consumers in ways that are convenient to see. For example, it is common practice to provide instructions on tampon use on leaflets inside a tampon package. The instructions may not be easy to access because the leaflet must be unfolded many times. As such, in certain embodiments, the present invention provides one or more pseudomotives having instructions for use of a feminine hygiene article. Such instructions may visible as the package is viewed or upon opening, and in certain embodiments, may be more accessible to the consumer and may be more likely to be seen and understood.

Section A below describes terms for assisting the reader in understanding features of the application, but not introducing limitations in the terms that may be inconsistent with the context in which they are used in the specification herein. Section B is the detailed description of the drawings illustrating a feminine hygiene applicator and other packaging elements in accordance with the embodiments of this application. Section C describes pseudomotives in general. Section D describes lenticular images. Section E describes holographic images. Section F describes in detail various embodiments of the use of pseudomotives as applied to consumer products.

A. Terms

As used herein, the term “feminine hygiene article” refers to articles that typically can be intended for feminine use, such as, e.g., sanitary napkins, liners, tampons, interlabial articles, incontinence articles, and pessaries.

As used herein, the term “tampon” refers to any type of absorbent structure such as, e.g., an absorbent mass, that can be inserted into the vaginal canal or other body cavity for the purpose of, such as, e.g., absorbing fluid, aiding in wound healing, and/or delivering materials, such as moisture or active materials such as medicaments. The term “tampon” can also include the combination of an absorbent structure with any type of applicator that can be associated with the absorbent structure to facilitate insertion of a tampon into the vaginal canal or other body cavity. A tampon can include any known tampon configuration such as, for example, digital tampons, tampons with traditional plunger type applicators, and/or tampons with compact applicators, such as, e.g., tampons described in U.S. Pat. Nos. 4,726,805; 4,846,802; 4,960,417; 5,087,239; 5,279,541; 6,258,075; 6,478,763; or any other known tampon.

The term “digital tampon” refers to a tampon that is intended to be inserted into the vaginal canal with the user's finger and without the aid of an applicator. Thus, digital tampons are typically visible to the consumer prior to use rather than being housed in an applicator.

As used herein, the terms “vaginal cavity” and “within the vagina” are intended to be synonymous and refer to the internal genitalia of the human female in the pudendal region of the body.

As used herein, the term “applicator” refers to a device or implement that facilitates the insertion of a tampon, medicament, treatment device, visualization aid, or other into an external orifice of a mammal, such as the vagina, rectum, ear canal, nasal canal, or throat.

As used herein, the term “tampon applicator” refers to an applicator for inserting a tampon. Non-limiting specific examples of such include any known hygienically designed applicator that is capable of receiving a tampon and may be used for the insertion of a tampon, including so-called telescoping tube and plunger designs and compact applicators.

As used herein, the term “pseudomotive” refers to a multidimensional image. Pseudomotives include, for example, holographic images and/or lenticular images. In certain embodiments, the pseudomotive can change from one image to a second image, such as, e.g., by flipping, morphing, zooming, and/or providing motion. In addition, or alternatively, the pseudomotive can include one or more image elements to provide a three-dimensional effect.

As used herein, the term “flip” refers to a perceived substitution of a second image for a first image as the viewing angle of an image changes. The images may be visually related or may be visually unrelated. In certain embodiments, flipping may be used in association with two or more images that are visually unrelated but are related in terms of the subject matter. As a non-limiting example, an image of a caterpillar may flip to an image of a butterfly as the viewing angle changes. Flipping may occur with left to right movement and vice versa. Flipping also may occur with top to bottom movement and vice versa.

As used herein, the term “morphing” refers to a perceived substitution of images having similar shapes and color densities but differing specific content in whole or in part. An image may appear to transition or undergo a metamorphous to another image when “morphing.”

As is used herein, term “motion” refers to the perception that a viewed image moves as the viewing angle of the observer changes. Non-limiting examples of motion include, without being limiting, the sweep of the beam of a lighthouse following the viewing angle of the observer, a bird in flight, a butterfly flapping its wings, a anthropomorphic character acting out a sequence of movements, a vehicle moving across the image, and combinations of such.

As is used herein, the term “zoom” refers to a shift in position of at least a portion of the image from the background of the image to the foreground of the image. The zoomed portion may appear to enlarge as the shift from the background to the foreground is achieved.

As is used herein, the term “depth of field” refers to a perception of three-dimensionality of an image wherein respective image elements are perceived to have a front to back spatial appearance. The image may be perceived to include distinct foreground elements and other distinct middle ground elements. The image may be perceived to include background elements as well.

As described above, these defined visual attributes may be perceived in combination. Motion and zoom, depth of field and motion, zoom and depth of field are non-limiting examples of possible combinations of image attributes. For example, an image element may be perceived as moving from the back of the image to the front of the image. An image element may be perceived as moving in the middle ground of the image behind foreground elements and in front of background elements. An image element may be perceived as moving from a first background scene to a second background scene.

B. General Description of a Tampon Applicator and Packaging

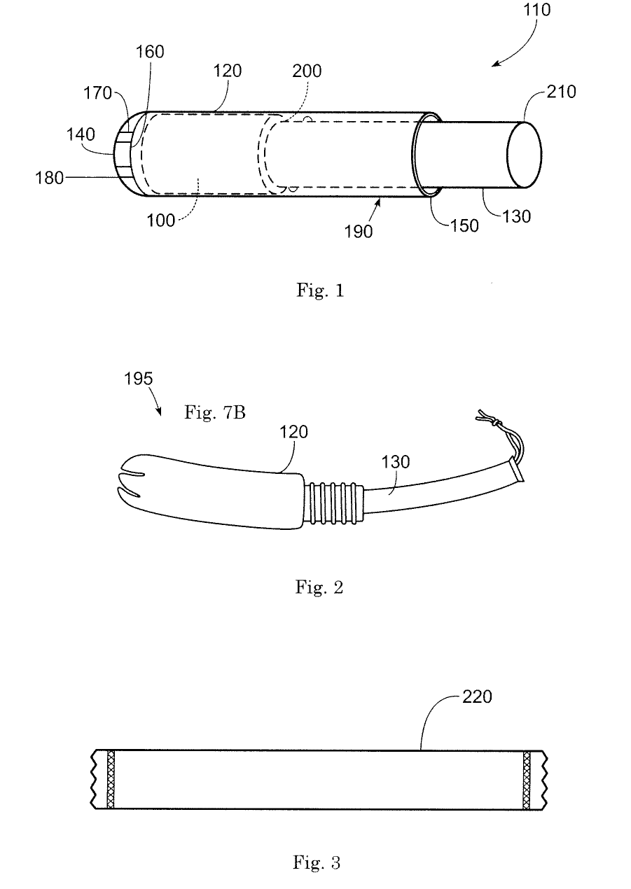

Referring now to the drawings in which like numerals refer to like elements throughout the several views, FIG. 1 shows a tampon 100 positioned within a tampon applicator 110. Although a tampon 100 and an applicator 110 are described herein, any type of feminine hygiene or other type of consumer product may be used herein. The tampon applicator 110 may be used with any type of tampon 100. The tampon applicator 110 may be made from rolled cardboard or other types of flushable, degradable materials. The tampon applicator 110 also may be made from polymeric material such as one or more plastics. Such applicators 110 generally may be opaque, all or partially transparent, and/or may be any color or colors. Any suitable size or shaped applicator may be used herein. The tampon applicator 110 may have graphics or other types of communications thereon.

In certain embodiments, the tampon applicator 110 may include an outer member 120 and an inner member 130. The outer member 120 may include an insertion end 140 and a second end 150 opposed to the insertion end 140. In certain embodiments, a preformed hinge or groove 160 may extend around the periphery of the outer member 120 near the insertion end 140. In addition, or alternatively, the outer member 120 also may have a domed shaped end having a number of radial slits 170 extending from a central aperture, such as, e.g., to the groove 160. In certain embodiments, the portion of the outer member 120 adjacent to the insertion end 140 also may have an openable end and may include a number of petals 180. The insertion end 140 may be opened in whole or in part. The outer member 120 also may have a grip region 190 positioned about the second end 150.

The inner member 130 may be dimensioned to slide within the outer member 120, such as, e.g., with minimal clearance therebetween. The inner member 130 may have a first end 200 and a second end 210. The first end 200 provides the necessary structure to expel the tampon 100 from the outer member 120. The inner member 130 may be solid or hollow in whole or in part. Other types of tampon applicators 110 may be used herein. Likewise, FIG. 2 shows a plastic applicator 195 also with an outer member 120 and an inner member 130. The applicator may be a molded plastic element or may be made by other techniques.

FIG. 3 shows a wrapper 220 for enclosing with the tampon 100 and the applicator 110. The wrapper 220 may made out of paper or similar materials although any type of material may be used herein. The tampon 100 and the applicator 110 generally may be individually wrapped within the wrapper 220 although other configurations may be used herein. The wrapper 220 may have any desired size or shape. The wrapper 220 may have graphics or other types of communication positioned thereon.

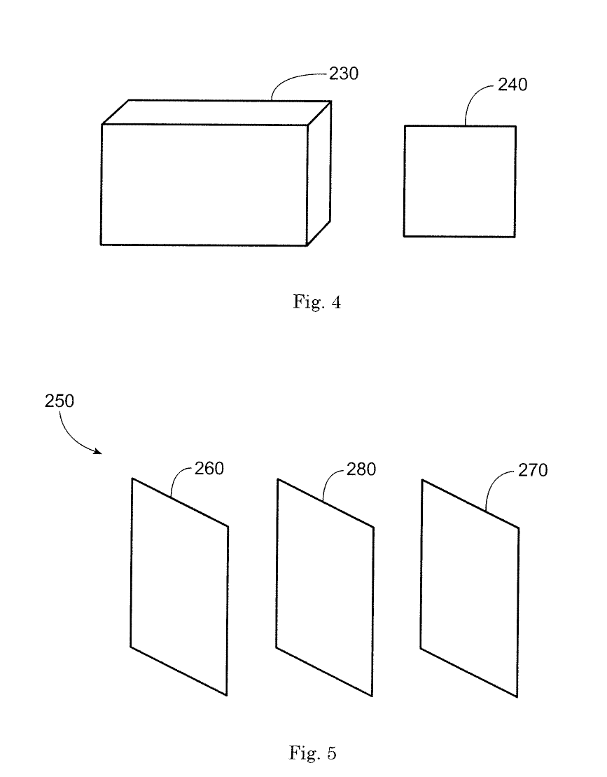

FIG. 4 shows a package 230 for enclosing the tampon 100 and the applicator 110. The package 230 may be a conventional box of any shape or size and may be made out of paperboard, cardboard, or any conventional type of material. In certain embodiments, the package 230 can be a bag, such as, e.g., a plastic bag, or any other suitable package 230. The package 230 may be constructed and enclosed by any known means. Any number of tampons 100 and applicators 110 may be positioned within the package 230. The package 230 may have graphics or other types of communication thereon.

A packaging insert 240 also may be positioned within the package 230. The packing insert 240 may be made out of paper or any other type of material. The insert 240 may include one or more communications to the consumer. These communications may include instructions for use, instructions for disposal, safety precautions or warnings, product information, advertising, coupons, or any other desired type of communication. Any number of inserts 240 may be used.

C. Pseudomotives

A number of different pseudomotives 245 are described herein. In certain embodiments, the pseudomotive 245 can include a lenticular pseudomotive 245 and/or a holographic pseudomotive 245 as described in more detail below. Other suitable pseudomotives 245 may be used herein. For example, commonly owned U.S. Patent Application Publication No. 2005/0095400, describes a “THREE-DIMENSIONAL PRODUCT WITH DYNAMIC VISUAL IMPACT.”

Similar processes may include other types of lenses, and other types of light channeling techniques. Further, three dimensional film technology also may be available from, e.g., Azuna LLC of Jenkintown, Pa. These films may mimic a holographic image with a series of raised bumps (Lencells) without the need for holographic photography. Moreover, several three-dimensional layers may be positioned on a poly-based substrate as opposed to a metal film. Three dimensional surfaces with heat activated textured inks may be available from Altivity Packaging LLC of Middletown, Ohio while three dimensional surfaces with laser expandable ink may be available from Siegwerk Ink Packaging of Des Moines, Iowa.

The pseudomotive 245 can include any suitable number of images. In certain embodiments, the pseudomotive 245 can have at least two images disposed thereon. For example, the pseudomotive 245 can have at least about two images, at least about three images, at least about four images, at least about five images, at least about six images, at least about seven images, at least about eight images, at least about nine images, at least about ten images, or any other suitable number of images disposed thereon. In certain embodiments, the pseudomotive 245 can have from about 2 to about 20 images, from about 2 to about 15 images, from about 2 to about 10 images, and/or from about 2 to about 5 images. In certain embodiments, the pseudomotive 245 can have more than about 20 images.

D. Lenticular Images

A lenticular image 250, such as, e.g., a label or film, is shown in FIG. 5. The lenticular image 250 as a whole may be made out of a thermoplastic material, for example, one or more of polyester, vinyl, polycarbonate, polyvinyl chloride (“PVC”), polyethylene terephthalate (“PET”), amorphous polyethylene terephthalate (“APET”), and similar types of materials. The lenticular image 250 also may be made out of biodegradable materials such as cellulose, cellulose acetate, polylactate acid (“PLA”), and similar types of materials.

In certain embodiments, the lenticular image 250 can include a film 260 and a lenticular lens 270. A communication 280 may be printed upon the film 260. The communication 280 may be any image desirably viewed through the lenticular lens 270. The communication 280 may be printed in a lenticular format. An exemplary embodiment of a lenticular communication 280 is created using a lenticular software tool. In certain embodiments, the lenticular software generally interlaces the desired images by cutting the images into narrow strips and then interlacing the strips in the appropriate orientation for viewing through the lenticular lens 270. For example, if two images are being interlaced, the software may alternate strips from the first and second images. Any method of creating a lenticular communication 280 is contemplated herein.

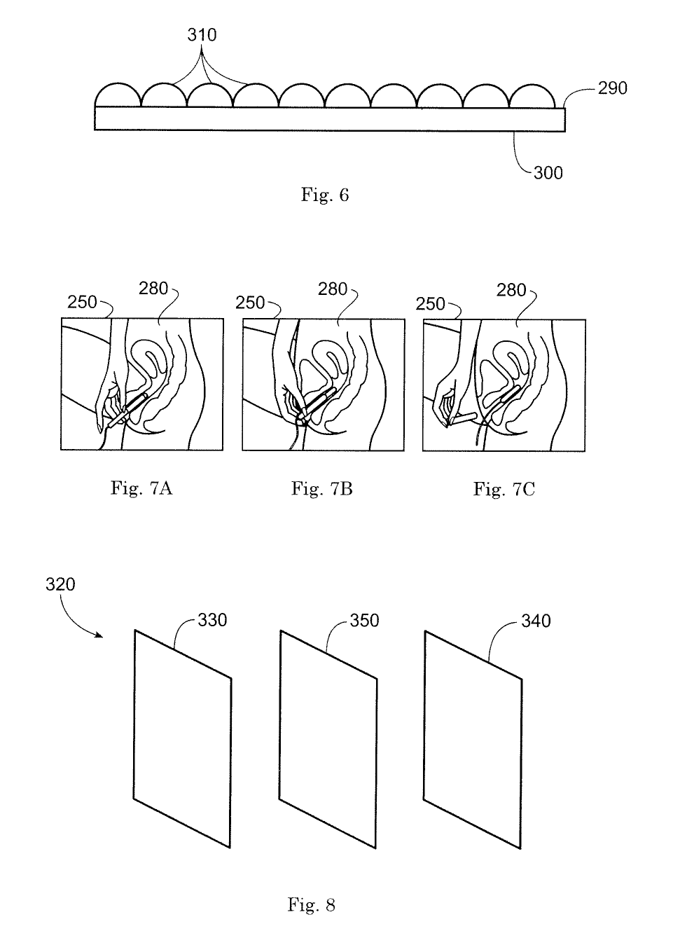

The film 260 may be applied to the lens 270. As is shown in FIG. 6, in certain embodiments, the lens 270 includes a first surface 290 and a second surface 300. The lenticular lens 270 may include a number of individual lenses 310 that project from the first surface 290 of the lenticular lens 270. The individual lenses 310 may have any shape that allows the viewer to view different images based upon the angular perspective, including, but not limited to, cylindrical, fly's eye, and other configurations. The second surface 300 over the lens 270 may be substantially flat for receiving the film 260. The individual lenses 310 may extend either vertically or horizontally or in any other desired orientation. The individual lenses 310 may be oriented in any density. The density of the lenses 310 should correspond with the strips of the communication 290 such that the viewer has the ability to see multiple images at different angular perspectives. The lenses 310 may have the density denominated in lenticules per inch (“LPI”) and may run from about 40 to about 240 LPI. Other densities may be used herein.

The communication 280 may include any combination of graphics, logos, text, or other type of visual information. The image provided by the label 250 can impart a visual illusion such as depth and/or motion to a viewer. The illusions may include “depth,” “motion” (including parallax motion), “flipping,” “zooming,” “morphing,” etc. As described above, a visual illusion of “depth” provides an image being viewed to appear as if it is in three-dimensions. A visual illusion of “motion” provides an image to appear as if it is undergoing movement. A visual illusion of “flipping” provides an image to appear as if it abruptly changes its orientation or as if it disappears and reappears. A visual illusion of “zooming” provides an image to appear as if it changes size, moving either closer or away to a viewer. A visual illusion of “morphing” provides an image of to appear as if it transitions or undergoes a metamorphous to another image. Generally, a multi-dimensional image may impart one or more visual illusions as may be desired. An example of a communication is shown in FIGS. 7A-7C in which instructions for use are shown with movement of the tampon 100 indicated.

A further alternative may be to place the communication 280 into motion separate and apart from the movement of the consumer. For example, the lenticular image 250 may be attached to a product and the product may be rotated, pivoted, or otherwise placed in motion such that the appearance and nature of the communication 280 varies even if the consumer is remains stationary. Likewise, the product or products may have multiple lenticular labels or film 250 thereon so as to provide multiple communications 280.

An example of a lenticular image 250 is shown in commonly owned U.S. Patent Application Publication No. 2005/0218025 A1, entitled “A PACKAGE ARTICLE COMPRISING A LENTICULAR LABEL.” Similar images are also described in commonly owned U.S. Patent Application Publication No. 2006/0131199 A1, entitled “CONSUMER PACKAGE HAVING VIEWING ANGLE DEPENDENT APPEARANCE.”

E. Holographic Images

The pseudomotive can also or alternatively include a holographic image 320. In certain embodiments, the holographic image 320 can be a label or film. As is shown in FIG. 8, in certain embodiments, a holographic image 320 can include an embossed layer 330 and a metallic layer 340. The holographic image 320 may provide a three-dimensional structural effect to the consumer. A communication 350 may be printed or applied to the embossed layer 330 prior to embossment. The communication 350 may be similar to the communication 280 described above. The embossed layer 330 can be a thermoplastic such as a polyester film. The embossed layer 330 may be embossed and the metallic layer 340, such as, e.g., aluminum, may be vaporized or applied in another manner onto the embossed layer 330 to form the holographic image 320. No particular manufacturing order may be required. Additional films or layers also may be applied. As described above, holographic labels or film 320 also may be made with a process of creating clear bumps over an image to cause them to lift off of the surface. Other types of materials and manufacturing methods may be used herein.

The holographic image 320 may provide the communication 350 as a three-dimensional image in whole or in part. Specifically, the image provided by the holographic image 320 may impart a visual illusion such as depth and/or motion to a viewer. Multiple images and/or communications 350 may be used herein.

A further alternative may be to place the communication 350 into motion separate and apart from the movement of the consumer. For example, the holographic image 320 may be attached to a product and the product may be rotated, pivoted, or otherwise placed in motion such that the appearance and nature of the communication 350 varies even if the consumer is remains stationary. Likewise, the product or products may have multiple lenticular labels or film 320 thereon so as to provide multiple communications 350.

An example of the holographic image 320 is shown in commonly owned U.S. Patent Application Publication No. 2004/0126494 A1 entitled “DUPLEX HOLOGRAPHIC FILM.” Similar types of holographic labels and films may be used herein.

F. Multidimensional Products and Packaging

Pseudomotives can be provided on any suitable product, packaging, advertisement, instruction sheet, insert, mailer, or any other suitable substrate. In certain embodiments, a pseudomotive can be provided at various locations relative to a feminine hygiene product, such as, e.g., at a point of purchase of the product, on the package, on an instruction sheet, and/or on the product itself, such as, e.g., as described herein. In addition, in certain embodiments, different pseudomotives can be provided at various locations relative to a feminine hygiene product, such as, e.g., a pseudomotive communicating absorption and/or flexibility can be provided at a point of purchase of the product and a pseudomotive communicating instructions for use and/or application of the feminine hygiene product can be provided on the product and/or an instruction sheet. In certain embodiments, a series of pseudomotives can be provided, such as, e.g., where each pseudomotive includes multiple images.

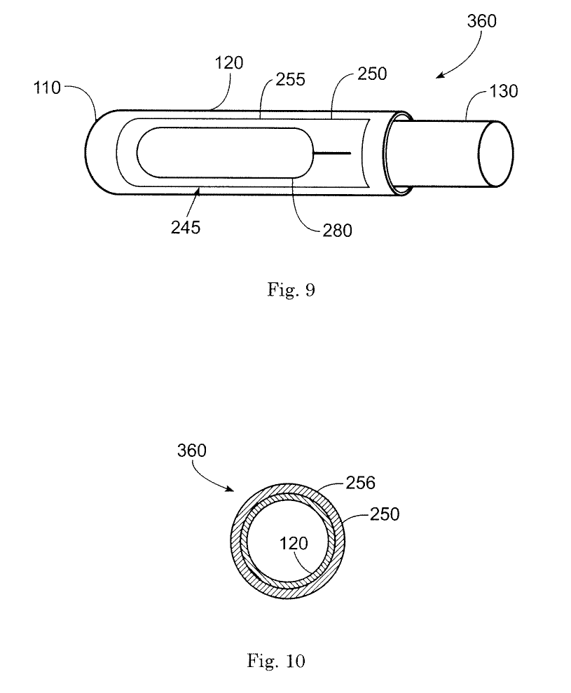

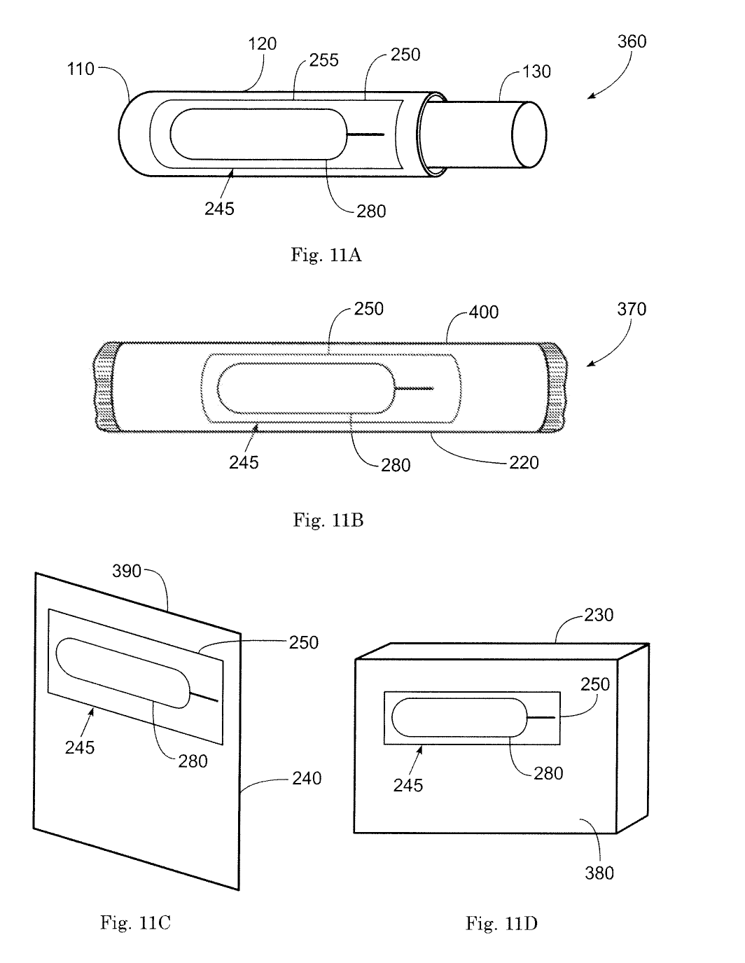

FIGS. 9 and 10 show embodiments of a lenticular applicator 360. Specifically, FIG. 9 shows the lenticular applicator 360 with one or more lenticular labels 255 positioned on the applicator 110. The lenticular labels 255 may be attached via an adhesive or via any conventional means. Alternatively, FIG. 10 shows a lenticular film 256 surrounding the applicator 110 in part or in whole. The lenticular film 256 may be applied as an applique, as a roll stock element, or any other type of suitable joinder means.

The lenticular applicator 360 may have one or more communications 280 positioned thereon. For example, one of the communications 280 may be an image of the tampon 100 itself. Moreover, the image of the tampon 100 may appear from a blank background, rotate, or even indicate the motion or direction of insertion. Likewise, through motion, multiple communications 280 may be conveyed to the consumer, including specific product functionality or benefits. The communication 280 may highlight features such as e.g., protection features, comfort features, absorbency, fit, strength, softness, performance, texture, “new and improved”, instructions for use, and/or any other desired message. Through the use of the lenticular image 250, the communication 280 may convey more information to the consumer as compared to conventional two-dimensional graphics.

FIG. 11 shows a lenticular kit 370. In certain embodiments, the lenticular kit 370 can include the lenticular applicator 360 as well as a lenticular package 380. The lenticular package 380 may include the package 230 with a lenticular image 250 positioned thereon. The lenticular package 380 also can include one or more communications 280 thereon. The communication 280 on the lenticular package 380 may be complementary or alternative to the communication 280 on the lenticular applicator 360. For example, in certain embodiments, it may be desired to have a common theme on both the lenticular applicator 360 and the lenticular package 380. Likewise, the image of the tampon 100 may be used on both the lenticular applicator 360 and the lenticular package 380. The lenticular kit 370 thus may effectively highlight a key message or design feature that is complemented or supplemented through the combination of the lenticular applicator 360 and the lenticular package 380.

The lenticular kit 370 also may include a lenticular insert 390 and/or a lenticular wrapper 400. The lenticular insert 390 may include the insert 240 with the lenticular image 250 thereon. The lenticular insert 390 can supplement the information on the insert 390. The lenticular wrapper 400 includes the wrapper 220 with the lenticular image 250 thereon. The lenticular wrapper 400 also may be made out of only the lenticular film 256. Both the lenticular insert 390 and the lenticular wrapper 400 may include one or more communications 280 thereon. As above, the communications 280 may be complementary or alternative as may be desired.

Specifically, the communications 280 may be used to educate the consumer as to the proper use of the tampon 100. The communication 280 may be used to encourage consumers to approach the lenticular package 380 and to remove the lenticular applicator 360 from the lenticular package 380 by illustrating motion that is related to the removal. The communication 280 may illustrate an upward motion on the lenticular package 380. The communication 280 also may relate to a product attribute to be desired by the consumer. The communication 280 may change in appearance depending upon the proximity of the observer to the lenticular package 380. The communication 280 may change as the observer approaches the lenticular package 380. This change may reinforce the behavior of approaching the lenticular package 380. A second communication 280 may encourage withdrawal of the lenticular applicator 360 from the lenticular package 380 for subsequent use. Any other type of communication 280 may be used herein. For example, a communication 280 depicting insertion directions located inside the lid of the package 230.

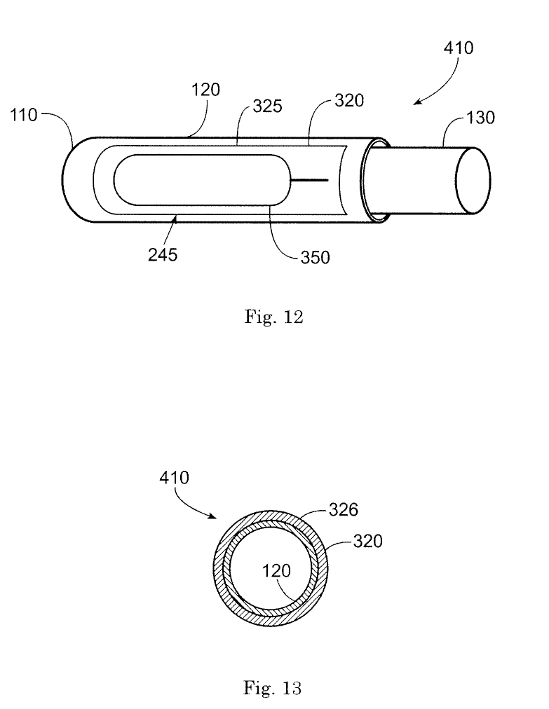

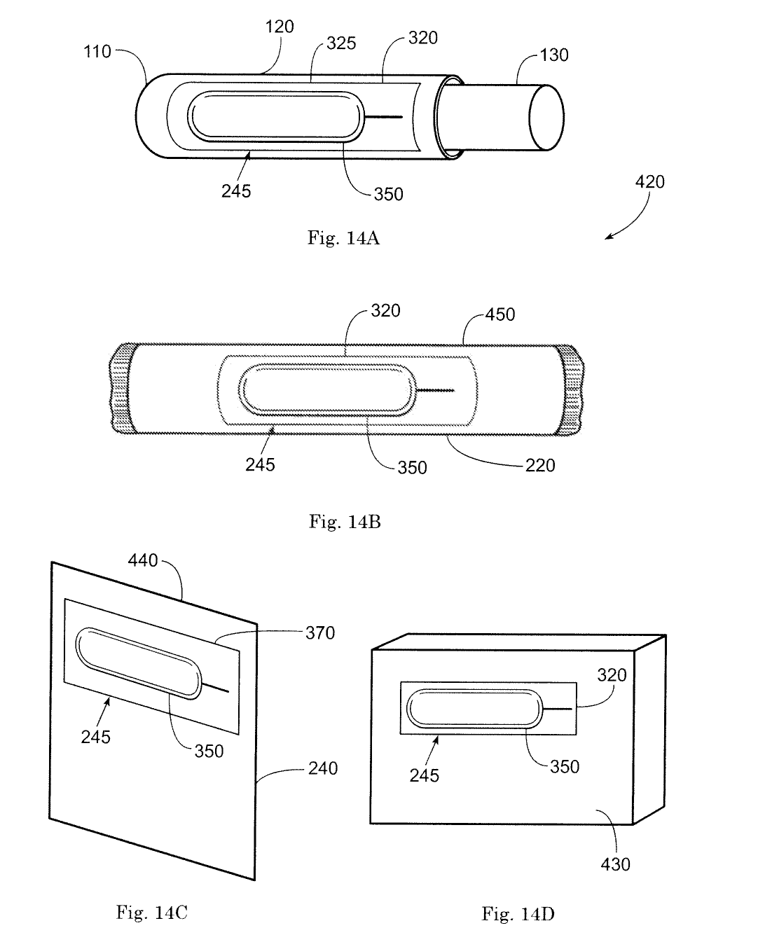

FIGS. 12 and 13 show embodiments of a holographic applicator 410. Specifically, FIG. 12 shows a holographic applicator 410 with one or more holographic labels 325 positioned on the applicator 110. The holographic labels 325 may be attached via an adhesive or via any conventional joinder means. FIG. 13 shows a holographic film 326 surrounding the applicator 110 in part or in whole. The holographic film 326 may be applied as an applique, as a roll stock element, or any other type of conventional joinder means.

The holographic applicator 410 may have one or more communications 350 positioned thereon. For example, one of the communications 350 may be a three-dimensional image of the tampon 100 itself. Likewise, multiple communications 350 may be conveyed, including specific product functionality or benefits. The communication 350 may highlight features such as e.g., protection features, comfort features, absorbency, fit, strength, softness, texture, performance, “new and improved”, instructions for use, and/or any other desired message. Through the use of the holographic image 320, the communication 350 may convey more information to the consumer as compared to conventional two-dimensional graphics.

FIG. 14 shows a holographic kit 420. As shown in FIG. 12, the holographic kit 420 may include the holographic applicator 410 as well as a holographic package 430. The holographic package 430 can include the package 230 with a holographic image 320 thereon. The holographic package 430 also can include one or more communications 350 thereon. The communication 350 on the holographic package 430 may be complementary or alternative to the communication 350 on the holographic applicator 410. For example, it may be desired to have a common theme on both the holographic applicator 410 and the holographic package 430. For example, the image of the tampon 100 also may be used on the holographic package 430. The holographic kit 420 thus may effectively highlight a key message or design feature that is complemented or supplemented through the combination of the holographic applicator 410 and holographic package 430.

The holographic kit 420 also may include a holographic insert 440 and/or a holographic wrapper 450. The holographic insert 440 can include the insert 240 with the holographic image 320 thereon. The holographic wrapper 450 can include the wrapper 220 with the holographic image 320 thereon. The holographic wrapper 450 also may be made out of only the holographic film 326. Both the holographic insert 440 and the holographic wrapper 450 may include one or more communications 350 thereon. As above, the communications 350 may be complementary or alternative as may be desired.

Specifically, the communications 350 may be used to educate the consumer as to the proper use of the tampon 100. The communication 350 may be used to encourage consumers to approach the holographic package 430 and to remove the holographic applicator 410 from the holographic package 430 by a three dimensional image of the tampon. The communication 280 may illustrate an upward motion on the holographic package 380. The communication 350 also may relate to a product attribute to be desired by the consumer. The communication 280 may change in appearance depending upon the proximity of the observer to the holographic package 430. The communication 350 may change as the observer approaches the holographic package 430. This change may reinforce the behavior of approaching the holographic package 430. A second communication 350 may encourage withdrawal of the holographic applicator 410 from the holographic package 430 for subsequent use. Any other type of communication 350 may be used herein.

In addition to the use of the lenticular image 250 or the holographic image 320, a lenticular or holographic structure also could be molded into an applicator 110. In the case of a plastic applicator 110, the lenticular or holographic structure could be molded into the applicator 110 via a two-stage molding process. Other types of molding or other manufacturing techniques may be used herein.

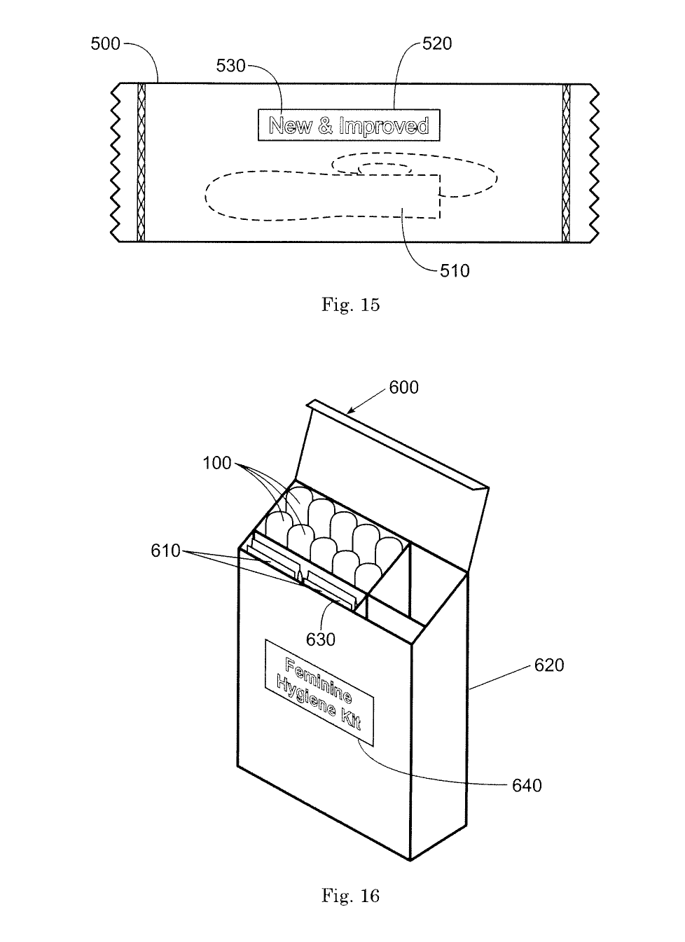

FIG. 15 shows a tampon wrapper 500. In certain embodiments, the tampon wrapper 500 may have a digital tampon 510 positioned therein. The tampon wrapper 500 may be made out of a substantially transparent or translucent material although any material suitable for the hygienic packaging of the tampon 510 may be used. The tampon wrapper 500 may have a tampon label 520 with a multidimensional communication 530 thereon. The multidimensional communication 530 may have lenticular, holographic, or any other type of pseudomotive thereon. Alternatively, the tampon wrapper 500 may be made out of a multidimensional film itself with the multidimensional communication 530 positioned thereon. The multidimensional communication 530 may be similar to the communications described above.

The lenticular label or film and the holographic image 320 also may be used in combination. Multiple or complimentary communications 280, 350 may be used. Although the present application has been described in terms of the applicators 360, 410 and the kits 370, 420, the techniques described herein are applicable to any type of consumer goods or packaging. The applicator 360, 410 and the kits 370, 420 are only one nonlimiting example. Other examples include, but are not limited to, food products, pet products, personal care products, cleaning products, paper products including paper towels, facial tissue and bath tissue, automotive products, baby care products, feminine care products (including sanitary napkins), health care products, and combinations of these products.

For example, FIG. 16 shows a feminine hygiene kit 600. The feminine hygiene kit 600 may include a number of tampons 100 and/or a number of sanitary napkins 610 or other types of feminine hygiene products positioned within a box 620. Any type of product or combinations of products may be used herein. The sanitary napkins 610 may be positioned within a wrapper 630. One or more communications 640 may be positioned about the tampon 100, the sanitary napkins 610, the wrapper 630, the box 620, or otherwise. The communication(s) 640 may be similar to the communications 280, 350 described above.

The dimensions and values disclosed herein are not to be understood as being strictly limited to the exact numerical values recited. Instead, unless otherwise specified, each dimension is intended to be the recited value and a functionally equivalent range surrounding that value. For example, the dimension disclosed as “40 mm” is intended to mean “about 40 mm.”

All documents cited in the Detailed Description of the Invention are, in relevant part, incorporated herein by reference. The citation of any document is not to be construed that it is an admission that it is prior art with respect to the present invention. To the extent that any meaning or definition of a term in this document conflicts with any meaning or definition of the same term in a document incorporated by reference or otherwise related, the meaning or definition assigned to this term in the document shall govern.

Although particular embodiments of the present invention have been illustrated and described, it would be obvious to one skilled in the art that various other changes and modifications may be made herein without departing from the general spirit and scope of the invention. It is therefore intended to cover in the independent claims all such changes and modifications that are within the scope of the invention and equivalents thereof.

We claim:

1. A feminine hygiene package, comprising:
a feminine hygiene product;
a package; and
the package comprising a pseudomotive thereon.

2. The feminine hygiene package of claim 1, wherein the feminine hygiene product is a sanitary napkin, liner, tampon, interlabial article, incontinence article, and/or pessary.

3. The feminine hygiene package of claim 1, wherein the feminine hygiene package comprises a second pseudomotive thereon.

4. The feminine hygiene package of claim 1, wherein the package comprises an insert, wherein the insert comprises the pseudomotive thereon.

5. The feminine hygiene package of claim 1, wherein the pseudomotive includes a lenticular image or a holographic image.

6. The feminine hygiene package of claim 1, wherein the pseudomotive includes from about 2 to about 24 images.

7. A feminine hygiene product comprising a pseudomotive thereon.

8. The feminine hygiene product of claim 7, wherein the feminine hygiene product is a sanitary napkin, liner, tampon, interlabial article, incontinence article, and/or pessary.

9. The feminine hygiene product of claim 7, wherein the pseudomotive includes a lenticular image or a holographic image.

10. The feminine hygiene product of claim 7, wherein the pseudomotive includes from about 2 to about 24 images.

11. The feminine hygiene product of claim 7, wherein the feminine hygiene product includes an applicator, the applicator comprising:
a first part;
a second part; and
the pseudomotive is positioned on the first part.

12. The feminine hygiene product of claim 7, wherein the pseudomotive comprises a vision of the feminine hygiene product.

13. The feminine hygiene product of claim 7, wherein the pseudomotive comprises a vision of a use, an application, and/or an insertion of the feminine hygiene product.

14. A pseudomotive, comprising a label or film, the label or film comprising:
a first image of a feminine hygiene product; and
a second image of the feminine hygiene product;
wherein the first image and the second image are different.

15. The pseudomotive of claim 14, wherein the label or film is lenticular.

16. The pseudomotive of claim 14, wherein the label or film is holographic.

17. The pseudomotive of claim 14, wherein the pseudomotive provides instructions for use of the feminine hygiene product.

18. The pseudomotive of claim 14, wherein pseudomotive illustrates one or more attributes of the feminine hygiene product selected from the group consisting of: flexibility, function, softness, thickness, absorption, scent, fit, or combinations thereof.

19. The pseudomotive of claim 14, wherein the pseudomotive is provided at the point of sale of the feminine hygiene product.

20. The pseudomotive of claim 14, wherein the pseudomotive comprises a vision of a use, an application, and/or an insertion of the feminine hygiene product.
